(19) United States
(12) Patent Application Publication
(10) Pub. No.: US 2022/0142271 A1
(43) Pub. Date: May 12, 2022

(54) SAFETY SUCTION MASK AND METHOD OF USE

(71) Applicant: QUALITY ASPIRATORS, INC., Duncanville, TX (US)

(72) Inventors: Brent A. Downs, Fort Worth, TX (US); Corless W. Wiley, Duncanville, TX (US); Virgilio A. Gomez, JR., Cedar Hill, TX (US); John B. Fisher, Grand Prairie, TX (US)

(73) Assignee: QUALITY ASPIRATORS, INC., Duncanville, TX (US)

(21) Appl. No.: 17/519,845

(22) Filed: Nov. 5, 2021

Related U.S. Application Data
(60) Provisional application No. 63/110,696, filed on Nov. 6, 2020.

(51) Int. Cl. A41D 13/11; A61C 17/12

(52) U.S. Cl. CPC A41D 13/1161; A61C 17/12

(57) ABSTRACT

A mask comprising a shield forming a perimeter, one or more portal penetrations through the shield, one or more suction inlets coupled to the shield, one or more ear loops coupled to the shield and a manifold coupled to the suction inlets and configured to distribute suction force.

### RELATED APPLICATIONS

The present application claims priority to and benefit of U.S. Provisional Patent application 63/110,696, entitled “Safety Suction Mask and Method of Use,” filed on Nov. 6, 2020, which is hereby incorporated by reference for all purposes as if set forth herein in its entirety.

### TECHNICAL FIELD

The present disclosure relates generally to safety equipment, and more specifically to a safety mask that includes suction to remove aerosols and particulates.

### BACKGROUND OF THE INVENTION

Suction devices are used to remove unwanted liquids from a patient's mouth during oral surgery, but often fail to provide substantial levels of removal of aerosols and particulates.

### SUMMARY OF THE INVENTION

A mask comprising a shield forming a perimeter, one or more portal penetrations through the shield, one or more suction inlets coupled to the shield, one or more ear loops coupled to the shield and a manifold coupled to the suction inlets and configured to distribute suction force.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a front isometric view of a mask having a suction structure, in accordance with an example embodiment of the present disclosure;

FIG. 2 is a diagram of a rear isometric view of a mask having a suction structure, in accordance with an example embodiment of the present disclosure;

FIG. 3 is a diagram of a front view of a mask having a suction structure, in accordance with an example embodiment of the present disclosure;

FIG. 4 is a diagram of a side view of a mask having a suction structure, in accordance with an example embodiment of the present disclosure;

FIG. 5 is a diagram of a top view of a mask having a suction structure, in accordance with an example embodiment of the present disclosure; and

FIG. 6 is a diagram of a rear view of a mask having a suction structure, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

The present disclosure provides a safety suction mask that is configured to reduce the production of aerosols during a dental treatment. In one example embodiment, a silicon mask can be used by the patient to provide a physical barrier that can capture and confine aerosol particulates so that they can be removed by suction. In this example embodiment, the safety suction mask can include a suction device on both the right and left sides of the mask, as well as a cover that confines any aerosols that are generated during the dental treatment, such as cleaning, examination, extraction, dental fillings, bonding, orthodontics, dental crowns, oral and maxillofacial procedures, orthodontics, periodontal treatment, laser treatment or other suitable dental treatments. The aerosols are then removed using suction, such as through a high-volume evacuator line or other suitable suction devices, to send the aerosol to the sanitary sewer.

Aerosol reduction is a rising focus in the dentistry field to protect patients and staff from air-born infectious diseases, such as the flue, COVID-19 and other diseases. Even with standard suction devices, aerosol overspray can still escape before it is suctioned up, thus spreading throughout the room. The present disclosure provides a system and method that confines the overspray of aerosols so that they can be effectively suctioned. The extra step to confine aerosol so that it can be suctioned can further protect those working in a dental setting.

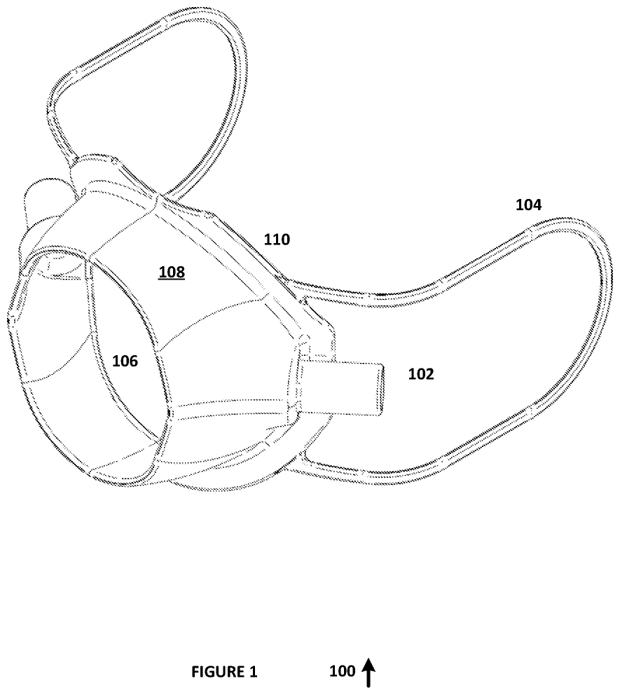

FIG. 1 is a diagram of a front isometric view of a mask 100 having a suction structure, in accordance with an example embodiment of the present disclosure. Mask 100 includes suction inlet 102, ear loop 104, portal 106, shield 108 and seal 110, each of which can be formed from plastic, silicone plastic, rubber, elastomeric or other suitable materials or combinations of materials, such as by extrusion molding, injection molding, compression molding, other suitable processes or a suitable combination of processes.

Suction inlet 102 is typical of two or more suction inlets to mask 100. In one example embodiment, suction inlet 102 can feed into shield 108 at a normal angle to a point of contact, can have a suitable angle of engagement, or other suitable configurations can also or alternatively be used. Suction inlet 102 can further include a manifold or other suitable structure that distributes the suction so as to cause a vortex effect or to otherwise improve the ability to remove aerosol particulates. While two suction inlets 102 are shown, a single suction inlet, three or more suction inlets or other suitable configurations of suction inlets can also or alternatively be provided. The suction inlets can be regularly or irregularly spaced around shield 108, can be diametrically opposed or disposed irregularly, such as to be located in a position where a patient is more likely to aspirate particles, such as based on salivary gland locations.

Ear loop 104 is typical of two ear loops, one on either side of mask 100, and can be configured to loop behind the ear of a user. In one example embodiment, ear loop 104 can be replaced with other suitable devices, such as a Velcro strap that meets with a mating Velcro strap behind the user's head. Ear loop 104 can be adjustable, formed from an elastic material or otherwise configured to facilitate use.

Portal 106 allows a dentist or other person to access the user's mouth, nose or other areas that are being treated or examined. In one example embodiment, portal 106 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 106 can have two or more different openings that are each covered with a replaceable film, where the user can remove a first film to access a first opening, and can then replace the first film and remove a second film to access a second opening.

Shield 108 extends around the perimeter of mask 100, such as to define opening 106 or to form other suitable structures. In one example embodiment, shield 108 allows suction inlets 102 to create a vortex to facilitate removal of aerosols. In another example embodiment, shield 108 can include one or more tool supports, suction supports to allow additional suction devices to be provided, blower supports to allow a source of compressed air to be provided to facilitate removal of aerosols or other suitable structures. Shield 108 also prevents non-aerosol particulates from being projected out of a patient's mouth or nose, such as by a rotary drill, a cautery device or other dental or surgical tools that generate non-aerosol particulates. Shield 108 can be flexible, such that portal 106 is not fixed in dimensions but rather flexes as needed.

Seal 110 couples to shield 108 and provides for a flexible seal against the face of a patient. In one example embodiment, seal 108 can be formed from a first material or covered with a first material that is different from the material of shield 108, such as a material that is hypoallergenic, a material that is more flexible or other suitable materials.

In operation, mask 100 provides for improved removal of aerosols during dental procedures, oral surgery, nasal surgery or other suitable procedures. Mask 100 reduces the discomfort that can be caused by suction devices that depend from a corner of the patient's mouth, provides more uniform suction and provides improved removal of aerosols and other particulates.

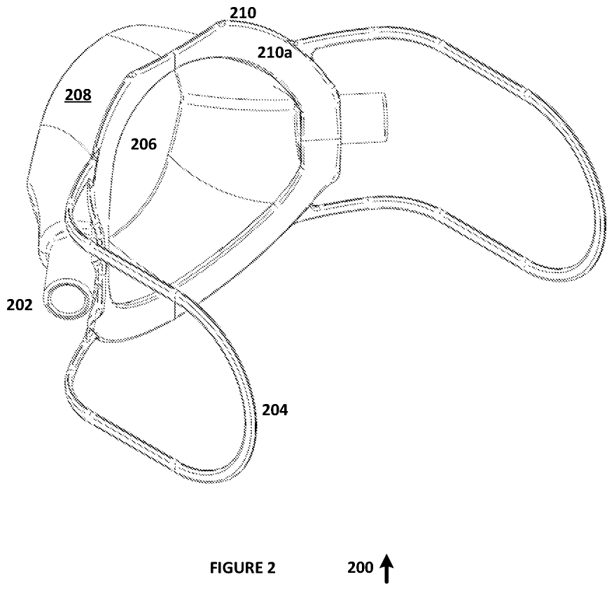

FIG. 2 is a diagram of a rear isometric view of a mask 200 having a suction structure, in accordance with an example embodiment of the present disclosure. Mask 200 includes suction inlet 202, ear loop 204, portal 206, shield 208, seal 210 and removable cover 210a, each of which can be formed from plastic, silicone plastic, rubber, elastomeric or other suitable materials or combinations of materials, such as by extrusion molding, injection molding, compression molding, other suitable processes or a suitable combination of processes.

Suction inlet 202 is typical of two or more suction inlets to mask 200. In one example embodiment, suction inlet 202 can feed into shield 208 at a normal angle to a point of contact, can have a suitable angle of engagement, or other suitable configurations can also or alternatively be used. Suction inlet 102 can further include a manifold or other suitable structure that distributes the suction so as to cause a vortex effect or to otherwise improve the ability to remove aerosol particulates.

Ear loop 204 is typical of two ear loops, one on either side of mask 200, and can be configured to loop behind the ear of a user. In one example embodiment, ear loop 204 can be replaced with other suitable devices, such as a Velcro strap that meets with a mating Velcro strap behind the user's head. Ear loop 204 can be adjustable, formed from an elastic material or otherwise configured to facilitate use.

Portal 206 allows a dentist or other person to access the user's mouth, nose or other areas that are being treated or examined. In one example embodiment, portal 206 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 206 can have two or more different openings that are each covered with a replaceable film, where the user can remove a first film to access a first opening, and can then replace the first film and remove a second film to access a second opening.

Shield 208 extends around the perimeter of mask 200, such as to define opening 206 or to form other suitable structures. In one example embodiment, shield 208 allows suction inlets 202 to create a vortex to facilitate removal of aerosols. In another example embodiment, shield 208 can include one or more tool supports, suction supports to allow additional suction devices to be provided, blower supports to allow a source of compressed air to be provided to facilitate removal of aerosols or other suitable structures. Shield 208 also prevents non-aerosol particulates from being projected out of a patient's mouth or nose, such as by a rotary drill, a cautery device or other dental or surgical tools that generate non-aerosol particulates. Shield 208 can be flexible, such that portal 206 is not fixed in dimensions but rather flexes as needed.

Seal 210 couples to shield 208 and provides for a flexible seal against the face of a patient. In one example embodiment, seal 208 can be formed from a first material or covered with a first material that is different from the material of shield 108, such as a material that is hypoallergenic, a material that is more flexible or other suitable materials. The covering material can be a thin film that is separate from the seal 210, such as removable covering 210a that can be placed on seal 210 or other surfaces of mask 200 after it has been sterilized, such as to allow a patient to see that the removable covering 210a has been in place immediately prior to being used or for other suitable purposes.

In operation, mask 200 provides for improved removal of aerosols during dental procedures, oral surgery, nasal surgery or other suitable procedures. Mask 200 reduces the discomfort that can be caused by suction devices that depend from a corner of the patient's mouth, provides more uniform suction and provides improved removal of aerosols and other particulates.

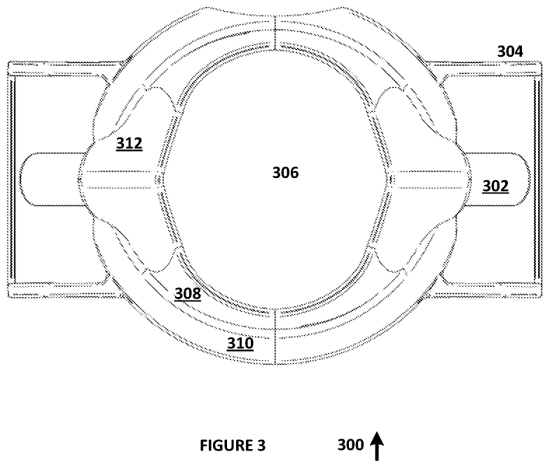

FIG. 3 is a diagram of a front view of a mask 300 having a suction structure, in accordance with an example embodiment of the present disclosure. Mask 300 includes suction inlet 302, ear loop 304, portal 306, shield 308, seal 310 and manifold 312, each of which can be formed from plastic, silicone plastic, rubber, elastomeric or other suitable materials or combinations of materials, such as by extrusion molding, injection molding, compression molding, other suitable processes or a suitable combination of processes.

Suction inlet 302 is typical of two or more suction inlets to mask 300. In one example embodiment, suction inlet 302 can feed into shield 108 at a normal angle to a point of contact, can have a suitable angle of engagement, or other suitable configurations can also or alternatively be used. Suction inlet 102 can further include a manifold or other suitable structure that distributes the suction so as to cause a vortex effect or to otherwise improve the ability to remove aerosol particulates.

Ear loop 304 is typical of two ear loops, one on either side of mask 300, and can be configured to loop behind the ear of a user. In one example embodiment, ear loop 304 can be replaced with other suitable devices, such as a Velcro strap that meets with a mating Velcro strap behind the user's head. Ear loop 304 can be adjustable, formed from an elastic material or otherwise configured to facilitate use.

Portal 306 allows a dentist or other person to access the user's mouth, nose or other areas that are being treated or examined. In one example embodiment, portal 306 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 306 can have two or more different openings that are each covered with a replaceable film, where the user can remove a first film to access a first opening, and can then replace the first film and remove a second film to access a second opening.

Shield 308 extends around the perimeter of mask 300, such as to define opening 306 or to form other suitable structures. In one example embodiment, shield 308 allows suction inlets 302 to create a vortex to facilitate removal of aerosols. In another example embodiment, shield 308 can include one or more tool supports, suction supports to allow additional suction devices to be provided, blower supports to allow a source of compressed air to be provided to facilitate removal of aerosols or other suitable structures. Shield 108 also prevents non-aerosol particulates from being projected out of a patient's mouth or nose, such as by a rotary drill, a cautery device or other dental or surgical tools that generate non-aerosol particulates. Shield 308 can be flexible, such that portal 306 is not fixed in dimensions but rather flexes as needed.

Seal 310 couples to shield 308 and provides for a flexible seal against the face of a patient. In one example embodiment, seal 308 can be formed from a first material or covered with a first material that is different from the material of shield 108, such as a material that is hypoallergenic, a material that is more flexible or other suitable materials.

Manifold 312 distributes suction around a periphery of mask 300. In one example embodiment, manifold 312 can be configured to increase the formation of a vortex to improve the removal of aerosols, particulates and other debris that can cause contamination.

In operation, mask 300 provides for improved removal of aerosols during dental procedures, oral surgery, nasal surgery or other suitable procedures. Mask 300 reduces the discomfort that can be caused by suction devices that depend from a corner of the patient's mouth, provides more uniform suction and provides improved removal of aerosols and other particulates.

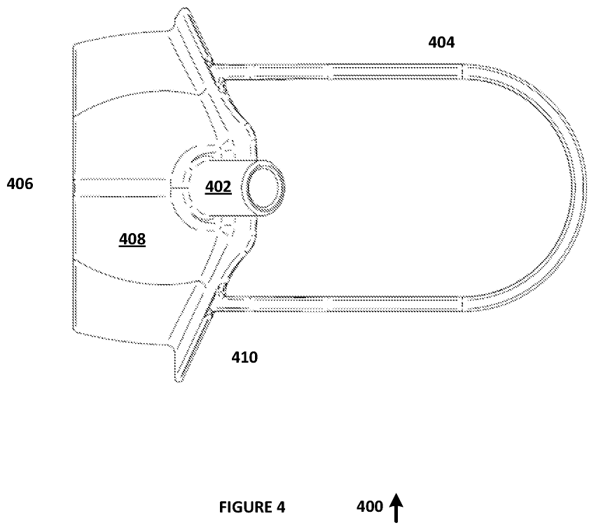

FIG. 4 is a diagram of a side view of a mask 400 having a suction structure, in accordance with an example embodiment of the present disclosure. Mask 400 includes suction inlet 402, ear loop 404, portal 406, shield 408 and seal 440, each of which can be formed from plastic, silicone plastic, rubber, elastomeric or other suitable materials or combinations of materials, such as by extrusion molding, injection molding, compression molding, other suitable processes or a suitable combination of processes.

Suction inlet 402 is typical of two or more suction inlets to mask 400. In one example embodiment, suction inlet 402 can feed into shield 408 at a normal angle to a point of contact, can have a suitable angle of engagement, or other suitable configurations can also or alternatively be used. Suction inlet 402 can further include a header or other suitable structure that distributes the suction so as to cause a vortex effect or to otherwise improve the ability to remove aerosol particulates.

Ear loop 404 is typical of two ear loops, one on either side of mask 400, and can be configured to loop behind the ear of a user. In one example embodiment, ear loop 404 can be replaced with other suitable devices, such as a Velcro strap that meets with a mating Velcro strap behind the user's head. Ear loop 404 can be adjustable, formed from an elastic material or otherwise configured to facilitate use.

Portal 406 allows a dentist or other person to access the user's mouth, nose or other areas that are being treated or examined. In one example embodiment, portal 406 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 406 can have two or more different openings that are each covered with a replaceable film, where the user can remove a first film to access a first opening, and can then replace the first film and remove a second film to access a second opening.

Shield 408 extends around the perimeter of mask 400, such as to define opening 406 or to form other suitable structures. In one example embodiment, shield 408 allows suction inlets 402 to create a vortex to facilitate removal of aerosols. In another example embodiment, shield 408 can include one or more tool supports, suction supports to allow additional suction devices to be provided, blower supports to allow a source of compressed air to be provided to facilitate removal of aerosols or other suitable structures. Shield 408 also prevents non-aerosol particulates from being projected out of a patient's mouth or nose, such as by a rotary drill, a cautery device or other dental or surgical tools that generate non-aerosol particulates. Shield 408 can be flexible, such that portal 406 is not fixed in dimensions but rather flexes as needed.

Seal 440 couples to shield 408 and provides for a flexible seal against the face of a patient. In one example embodiment, seal 408 can be formed from a first material or covered with a first material that is different from the material of shield 408, such as a material that is hypoallergenic, a material that is more flexible or other suitable materials.

In operation, mask 400 provides for improved removal of aerosols during dental procedures, oral surgery, nasal surgery or other suitable procedures. Mask 400 reduces the discomfort that can be caused by suction devices that depend from a corner of the patient's mouth, provides more uniform suction and provides improved removal of aerosols and other particulates.

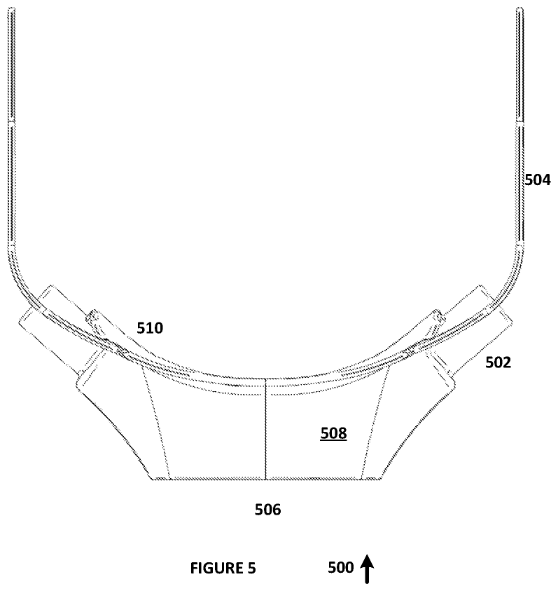

FIG. 5 is a diagram of a top view of a mask 500 having a suction structure, in accordance with an example embodiment of the present disclosure. Mask 500 includes suction inlet 502, ear loop 504, portal 506, shield 508 and seal 550, each of which can be formed from plastic, silicone plastic, rubber, elastomeric or other suitable materials or combinations of materials, such as by extrusion molding, injection molding, compression molding, other suitable processes or a suitable combination of processes.

Suction inlet 502 is typical of two or more suction inlets to mask 500. In one example embodiment, suction inlet 502 can feed into shield 508 at a normal angle to a point of contact, can have a suitable angle of engagement, or other suitable configurations can also or alternatively be used. Suction inlet 502 can further include a header or other suitable structure that distributes the suction so as to cause a vortex effect or to otherwise improve the ability to remove aerosol particulates.

Ear loop 504 is typical of two ear loops, one on either side of mask 500, and can be configured to loop behind the ear of a user. In one example embodiment, ear loop 504 can be replaced with other suitable devices, such as a Velcro strap that meets with a mating Velcro strap behind the user's head. Ear loop 504 can be adjustable, formed from an elastic material or otherwise configured to facilitate use.

Portal 506 allows a dentist or other person to access the user's mouth, nose or other areas that are being treated or examined. In one example embodiment, portal 506 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 506 can have two or more different openings that are each covered with a replaceable film, where the user can remove a first film to access a first opening, and can then replace the first film and remove a second film to access a second opening.

Shield 508 extends around the perimeter of mask 500, such as to define opening 506 or to form other suitable structures. In one example embodiment, shield 508 allows suction inlets 502 to create a vortex to facilitate removal of aerosols. In another example embodiment, shield 508 can include one or more tool supports, suction supports to allow additional suction devices to be provided, blower supports to allow a source of compressed air to be provided to facilitate removal of aerosols or other suitable structures. Shield 508 also prevents non-aerosol particulates from being projected out of a patient's mouth or nose, such as by a rotary drill, a cautery device or other dental or surgical tools that generate non-aerosol particulates. Shield 508 can be flexible, such that portal 506 is not fixed in dimensions but rather flexes as needed.

Seal 550 couples to shield 508 and provides for a flexible seal against the face of a patient. In one example embodiment, seal 508 can be formed from a first material or covered with a first material that is different from the material of shield 508, such as a material that is hypoallergenic, a material that is more flexible or other suitable materials.

In operation, mask 500 provides for improved removal of aerosols during dental procedures, oral surgery, nasal surgery or other suitable procedures. Mask 500 reduces the discomfort that can be caused by suction devices that depend from a corner of the patient's mouth, provides more uniform suction and provides improved removal of aerosols and other particulates.

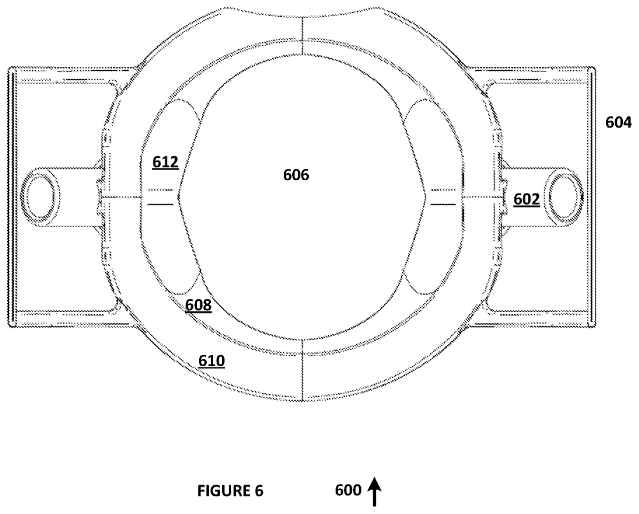

FIG. 6 is a diagram of a rear view of a mask 600 having a suction structure, in accordance with an example embodiment of the present disclosure. Mask 600 includes suction inlet 602, ear loop 604, portal 606, shield 608, seal 610 and manifold 612, each of which can be formed from plastic, silicone plastic, rubber, elastomeric or other suitable materials or combinations of materials, such as by extrusion molding, injection molding, compression molding, other suitable processes or a suitable combination of processes.

Suction inlet 602 is typical of two or more suction inlets to mask 600. In one example embodiment, suction inlet 602 can feed into shield 108 at a normal angle to a point of contact, can have a suitable angle of engagement, or other suitable configurations can also or alternatively be used. Suction inlet 102 can further include a manifold or other suitable structure that distributes the suction so as to cause a vortex effect or to otherwise improve the ability to remove aerosol particulates.

Ear loop 604 is typical of two ear loops, one on either side of mask 600, and can be configured to loop behind the ear of a user. In one example embodiment, ear loop 604 can be replaced with other suitable devices, such as a Velcro strap that meets with a mating Velcro strap behind the user's head. Ear loop 604 can be adjustable, formed from an elastic material or otherwise configured to facilitate use.

Portal 606 allows a dentist or other person to access the user's mouth, nose or other areas that are being treated or examined. In one example embodiment, portal 606 can be sized to allow the dentist or other person to perform predetermined activities, such as to provide a larger opening for activities that require a greater field of movement, a smaller opening for activities that do not require a large field of movement, one or more penetrations that allow specific tools to be inserted through the portal, an adjustable opening that allows the dentist or other person to select the size of the opening, or other suitable configurations. In one example embodiment, portal 606 can have two or more different openings that are each covered with a replaceable film, where the user can remove a first film to access a first opening, and can then replace the first film and remove a second film to access a second opening.

Shield 608 extends around the perimeter of mask 600, such as to define opening 606 or to form other suitable structures. In one example embodiment, shield 608 allows suction inlets 602 to create a vortex to facilitate removal of aerosols. In another example embodiment, shield 608 can include one or more tool supports, suction supports to allow additional suction devices to be provided, blower supports to allow a source of compressed air to be provided to facilitate removal of aerosols or other suitable structures. Shield 108 also prevents non-aerosol particulates from being projected out of a patient's mouth or nose, such as by a rotary drill, a cautery device or other dental or surgical tools that generate non-aerosol particulates. Shield 608 can be flexible, such that portal 606 is not fixed in dimensions but rather flexes as needed.

Seal 610 couples to shield 608 and provides for a flexible seal against the face of a patient. In one example embodiment, seal 608 can be formed from a first material or covered with a first material that is different from the material of shield 108, such as a material that is hypoallergenic, a material that is more flexible or other suitable materials.

Manifold 612 distributes suction around a periphery of mask 600. In one example embodiment, manifold 612 can be configured to increase the formation of a vortex to improve the removal of aerosols, particulates and other debris that can cause contamination.

In operation, mask 600 provides for improved removal of aerosols during dental procedures, oral surgery, nasal surgery or other suitable procedures. Mask 600 reduces the discomfort that can be caused by suction devices that depend from a corner of the patient's mouth, provides more uniform suction and provides improved removal of aerosols and other particulates.

In use, suction hoses can be coupled to masks 100 through 600 before or after the masks are affixed to a patient. The ear loops can be adjusted or tightened, where suitable, and the size of the portals or portal openings can be adjusted, where suitable. The suction can then be controlled, such as to increase suction when a powered tool is in operation by synchronizing the suction with the application of power to the tool, suction can be continuously applied or other suitable processes can also or alternatively be used. A suction device attached to the tool can also be used to increase aerosol or particulate removal, or a compressed air stream can be provided from an attachment to the tool or an independent compressed air source to improve the ability to remove aerosols and particulates. After use, the mask can be sterilized, one or more components of the mask can be replaced or other suitable procedures can be used to minimize the risk of disease transmission or contamination, such as destruction of the mask. The mask can also or alternatively have one or more components that dissolve in a predetermined solution, can have a coating that dissolves or can have other suitable features to facilitate decontamination processing.

In one example embodiment, an apparatus is disclosed that includes a shield such as a circular or other suitable shaped shield forming a perimeter, a first portal penetration on a first side of the shield, a second portal penetration on a second side of the shield and one or more suction inlets coupled to the shield so as to create a negative pressure inside of the shield relative to an area outside of the shield to prevent particulates that may be aspirated into the shield from existing the space contained by the shield between the portal penetrations.

The apparatus can comprise one or more ear loops coupled to the shield, so as to assist with holding the shield in position against a face of a patient, such as where the shield is being used during oral surgery, dental cleaning, a dental procedure such as a root canal, extraction, filling a cavity, installing a crown, installing a tooth veneer, implanting an artificial replacement for a tooth or performing other dental procedures.

The first portal penetration of the apparatus can be coupled to a seal that is configured to conform to a face of a user, such as a flexible seal that surrounds the exterior surface of the portal. The seal can be made of a suitable flexible material, such as rubber, a polymer, an elastomer, a composite material or other suitable materials.

The second portal penetration can be configured to provide access to a user's mouth during a dental procedure. For example, it can be smaller than the first portal penetration so as to allow enough room for one or more dental instruments while reducing the area from which particulates could be aspirated. The shield can be rigid, flexible, can have rigid portions that separate flexible portions or can use other suitable configurations.

The suction inlets can include two diametrically opposed suction inlets, such as a first suction inlet on a left side of a face of a user wearing the shield and a second suction inlet on a right side of the face of the user, or other suitable placements.

A manifold cam be coupled to one of the suction inlets and configured to distribute suction force within the shield. The manifold can extend around the interior of the shield, such as with a predetermined number of inlet ports that distribute the suction force or in other suitable manners. The one or more suction inlets are coupled to the shield between the first portal penetration and the second portal penetration.

The first portal penetration can further comprise a removable covering over a sanitized surface, which can be removed in front of the patient prior to placing the first portal penetration against the patient's face. The removable covering can cover as many sanitized surfaces as may be present on the apparatus, including but not limited to the entire apparatus where suitable. The apparatus can be reusable after sterilization, and a new removable covering can be placed on the apparatus after it has been used and sterilized.

A method for providing suction is also disclosed that includes placing a shield against a patient's face, the shield having a perimeter with first portal penetration on a first side of the shield, attaching one or more suction inlets of the shield to a suction source and applying suction during an oral procedure. The method can further comprise placing an ear loop coupled to the shield over an ear of the patient. The method can further comprise conforming a seal coupled to the first portal penetration to the patient's face. The method can further comprise performing a dental procedure on the patient through a second portal penetration and the first portal penetration. The method can further comprise removing a covering from the first side of the shield to expose a sanitized surface prior to placing the shield against the patient's face. The method can further comprise modulating the application of suction, such as to reduce the amount of drying that would otherwise occur to the patient's mouth if suction were continuously applied at a high rate. For example, when teeth are being cleaned, a rotary device can be used that causes particulates to be generated, and suction can be modulated to be increased when that rotary device is being operated. Likewise, suction can be modulated based on other procedures, such as to increase suction during periods where a procedure is being performed that generates particulates and to reduce suction when particulates are not being generated.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. An apparatus comprising:
a shield forming a perimeter;
a first portal penetration on a first side of the shield;
a second portal penetration on a second side of the shield; and
one or more suction inlets coupled to the shield.

2. The apparatus of claim 1 further comprising one or more ear loops coupled to the shield.

3. The apparatus of claim 1 wherein the first portal penetration is coupled to a seal that is configured to conform to a face of a user.

4. The apparatus of claim 1 wherein the second portal penetration is configured to provide access to a user's mouth during a dental procedure.

5. The apparatus of claim 1 wherein the shield is rigid.

6. The apparatus of claim 1 wherein the shield is flexible.

7. The apparatus of claim 1 wherein the one or more suction inlets comprise two diametrically opposed suction inlets.

8. The apparatus of claim 1 comprising a first suction inlet on a left side of a face of a user wearing the shield and a second suction inlet on a right side of the face of the user.

9. The apparatus of claim 1 comprising a manifold coupled to one of the suction inlets and configured to distribute suction force within the shield.

10. The apparatus of claim 1 wherein the one or more suction inlets are coupled to the shield between the first portal penetration and the second portal penetration.

11. The apparatus of claim 1 wherein the first portal penetration further comprises a removable covering over a sanitized surface.

12. A method for providing suction, comprising:
placing a shield against a patient's face, the shield having a perimeter with first portal penetration on a first side of the shield;
attaching one or more suction inlets of the shield to a suction source; and
applying suction during an oral procedure.

13. The method of claim 12 further comprising placing an ear loop coupled to the shield over an ear of the patient.

14. The method of claim 12 further comprising conforming a seal coupled to the first portal penetration to the patient's face.

15. The method of claim 12 further comprising performing a dental procedure on the patient through a second portal penetration and the first portal penetration.

16. The method of claim 12 further comprising removing a covering from the first side of the shield to expose a sanitized surface prior to placing the shield against the patient's face.

17. The method of claim 12 further comprising modulating the application of suction.

18. In an apparatus having a shield forming a perimeter, a first portal penetration on a first side of the shield, a second portal penetration on a second side of the shield, one or more suction inlets coupled to the shield, one or more ear loops coupled to the shield, wherein the first portal penetration is coupled to a seal that is configured to conform to a face of a user, wherein the second portal penetration is configured to provide access to a user's mouth during a dental procedure, wherein the shield is rigid or flexible, wherein the one or more suction inlets comprise two diametrically opposed suction inlets, a first suction inlet on a left side of a face of a user wearing the shield and a second suction inlet on a right side of the face of the user, a manifold coupled to one of the suction inlets and configured to distribute suction force within the shield, wherein the one or more suction inlets are coupled to the shield between the first portal penetration and the second portal penetration, and wherein the first portal penetration further comprises a removable covering over a sanitized surface, a method comprising:
placing a shield against a patient's face, the shield having a perimeter with first portal penetration on a first side of the shield;
attaching one or more suction inlets of the shield to a suction source;
applying suction during an oral procedure;
placing an ear loop coupled to the shield over an ear of the patient;
conforming a seal coupled to the first portal penetration to the patient's face;
performing a dental procedure on the patient through a second portal penetration and the first portal penetration;
removing a covering from the first side of the shield to expose a sanitized surface prior to placing the shield against the patient's face; and
modulating the application of suction.
